SEQUENCE LISTING (7 sequences)

SEQ ID NO: 1 - DNA, 23 bp, Artificial sequence
  Oligonucleotide primer
gactgaagagggcaatgttgagc

SEQ ID NO: 2 - DNA, 21 bp, Artificial sequence
  Modified_base 16: Oligonucleotide primer with y residue at position 16
gcaataactgcggacytctgc

SEQ ID NO: 3 - protein, 14 aa, Artificial sequence
  Polypeptide fragment of West Nile Virus E Protein
CRVKMEKLQLKGTT

SEQ ID NO: 4 - protein, 14 aa, Artificial sequence
  Polypeptide fragment of West Nile Virus E Protein
CQLLMREVKTGTKK

SEQ ID NO: 5 - protein, 19 aa, Artificial sequence
  Polypeptide fragment of West Nile Virus E Protein
CSTKAIGRTILKENIKYEV

SEQ ID NO: 6 - DNA, 27 bp, Artificial sequence
  Oligonucleotide primer
gaattcttcaactgccttggaatgagc

SEQ ID NO: 7 - DNA, 27 bp, Artificial sequence
  Oligonucleotide primer
ctgcagttatttgccaatgctgcttcc

(19) United States
(12) Patent Application Publication
(10) Pub. No.: US 2003/0148261 A1
(43) Pub. Date: Aug. 7, 2003

(54) Compositions and methods comprising West Nile virus polypeptides

(76) Inventors: Erol Fikrig, Guilford, CT (US); Raymond A. Koski, Old Lyme, CT (US); Tian Wang, New Haven, CT (US)

Correspondence Address: FISH & NEAVE, 1251 AVENUE OF THE AMERICAS, 50TH FLOOR, NEW YORK, NY, 10020-1105, US

(21) Appl. No.: 10/096,376

(22) Filed: Mar. 11, 2002

Related U.S. Application Data
(60) Provisional application No. 60/275,025, filed on Mar. 12, 2001.
(60) Provisional application No. 60/281,947, filed on Apr. 5, 2001.

(51) Int. Cl. C12Q 1/70; A61K 39/12; C12N 7/00; C07K 14/005

(52) U.S. Cl. 435/5; 424/204.1; 435/235.1; 530/350

(57) ABSTRACT

This application is directed to compositions and methods comprising isolated and purified West Nile virus polypeptides and immunogenic fragments thereof, nucleic acid molecules encoding them and antibodies specific for such polypeptides or fragments. Such polypeptides and fragments, fusion proteins comprising them and antibodies are useful as vaccines to treat, inhibit or prevent West Nile virus infection or disease, to detect West Nile virus infection and to monitor the course of disease or immunization.

### CROSS REFERENCE TO RELATED APPLICATIONS

This application claims benefit from U.S. Provisional Application No. 60/275,025, filed Mar. 12, 2001 and U.S. Provisional Application No. 60/281,947, filed Apr. 5, 2001, the disclosures of which are hereby incorporated by reference in their entireties.

### TECHNICAL FIELD OF THE INVENTION

This invention relates to compositions and methods for diagnosing West Nile (“WN”) virus infection, conferring immunity to WN virus and for the prevention of WN virus infections. More particularly, this invention relates to isolated and/or purified polypeptides (including recombinant, synthetic and fusion proteins comprising the polypeptides or synthetic peptides) from WN virus that are useful to detect WN virus infection in a subject, to nucleic acid molecules encoding the polypeptides and to pharmaceutical compositions comprising one or more polypeptides of the invention that elicit an immune response that is effective either to prevent WN virus infection or to significantly reduce morbidity and mortality from WN virus infection.

Also within the scope of this invention are antibodies that specifically bind a polypeptide, peptide or fusion protein of the invention and vaccines comprising one or more of the antibodies of this invention. Additionally, this invention includes diagnostic kits comprising one or more of the polypeptides, peptides or fusion proteins described in the invention or antibodies that specifically bind a polypeptide, peptide or fusion protein of the invention.

### BACKGROUND OF THE INVENTION

In the summer of 1999, an outbreak of encephalitis in humans that was associated with mosquitoes occurred in New York City [CDC, MMWR, 48, pp. 845-9 (1999); CDC, MMWR, 48, pp. 944-6 (1999); D. S. Asnis et al., Clin Infect Dis, 30, pp. 413-8 (2000)]. At approximately the same time, American crows began dying in the Northeastern United States, many in Fairfield County, Conn. Two reports in December of 1999 demonstrated that these outbreaks in birds and humans were actually due to WN virus transmitted by mosquitoes [R. S. Lanciotti et al., Science, 286, pp. 2333-7 (1999); J. F. Anderson et al., Science, 286, pp.2331-3 (1999)]. It is clear from these reports that WN virus was the cause of the 1999 outbreak of fatal encephalitis in the Northeastern United States. This is the first reported appearance of WN virus in the Western Hemisphere.

Future outbreaks of WN virus in the United States are a new and important public health concern. To date, the only method for preventing WN virus infection is spraying large geographic areas with insecticide to kill mosquito vectors. Spraying is difficult, potentially toxic to humans, requires repeated applications and is incompletely effective. There is no known vaccine against WN virus. Accordingly, there is an urgent need for a vaccine to prevent infection by WN virus.

West Nile virus is a member of the family Flaviviridae which also includes the Japanese encephalitis virus (JE), Tick-borne encephalitis virus (TBE), dengue virus (including the four serotypes of: DEN-1, DEN-2, DEN-3, and DEN-4), and the family prototype, yellow fever virus (YF). Flavivirus infections are a global public health problem [C. G. Hayes, in The Arboviruses: Epidemiology and Ecology, T. P. Monathy, ed., CRC, Boca Raton, Fla., vol. 5, chap. 49 (1989); M. J. Cardosa, Br Med Bull, 54, pp. 395-405 (1998); Z. Hubalek and J. Halouzka, Emerg Infect Dis, 5, pp. 643-50 (1999)] with about half of the flaviviruses causing human diseases. These viral pathogens are transmitted by mosquito or tick vectors. Birds, including the American crow, Corvus brachyrhynchos, can serve as non-human reservoirs for the virus. In the case of WN virus, the viruses are transmitted to man by mosquitoes and in the Northeastern United States these mosquito vectors are primarily of the genera Culex and Aedes, particularly C. pipiens and A. vexans.

Flaviviruses are the most significant group of arthropod-transmitted viruses in terms of global morbidity and mortality. An estimated one hundred million cases of the most prevalent flaviviral disease, dengue fever, occur annually. Flaviviral disease typically occurs in the tropical and subtropical regions. Increase global population and urbanization coupled with the lack of sustained mosquito control measures, has distributed the mosquito vectors of flaviviruses throughout the tropics, subtropics, and some temperate areas. As a result over half the world's population is at risk for flaviviral infection. Further, modern jet travel and human migration have raised the potential for global spread of these pathogens.

West Nile virus infections generally have mild symptoms, although infections can be fatal in elderly and immunocompromised patients. Typical symptoms of mild WN virus infections include fever, headache, body aches, rash and swollen lymph glands. Severe disease with encephalitis is typically found in elderly patients [D. S. Asnis et al., supra]. Death can result from effects on the central nervous system. Sixty-two severe cases and seven deaths were attributed to WN virus encephalitis during the 1999 outbreak [CDC, supra; CDC, supra; D. S. Asnis et al., supra]. Although most WN virus infections are mild, concern is particularly heightened by the potentially fatal outcome of this mosquito-transmitted disease.

The WN virus, like other flaviviruses, is enveloped by host cell membrane and contains the three structural proteins capsid (C), membrane (M), and envelope (E). The E and M proteins are found on the surface of the virion where they are anchored in the membrane. Mature E is glycosylated, whereas M is not, although its precursor, prM, is a glycoprotein. In other flaviviruses, glycoprotein E is the largest structural protein and contains functional domains responsible for cell surface attachment and intraendosomal fusion activities. In some flaviviruses, E protein has been reported to be a major target of the host immune system during a natural infection.

The flavivirus genome is a single positive-stranded RNA of approximately 10,500 nucleotides containing short 5′ and 3′ untranslated regions, a single long open reading frame (ORF), a 5′ cap, and a nonpolyadenylated 3′ terminus. The ten gene products encoded by the single, long ORF are contained in a polyprotein organized in the order, C (capsid), prM/M (membrane), E (envelope), NS1 (nonstructural protein 1), NS2A, NS2B, NS3, NS4A, NS4B, and NS5 [T. J. Chambers et al., Ann Rev Microbiol, 44, pp. 649-88 (1990)].

Viral replication occurs in the cytoplasm of the infected cell and processing of the encoded polyprotein is initiated cotranslationally. Full maturation of viral proteins requires both host and viral-specific proteases. The sites of proteolytic cleavage in the YF virus, which is likely to be predictive of the sites of cleavage in all flaviviruses, have been determined by comparing the nucleotide sequence and the amino terminal sequences of the viral proteins. Subsequent to initial processing of the polyprotein, prM is converted to M during virus release [G. Wengler at al., J Virol, 63, pp. 2521-6 (1989)], and anchored C is processed during virus maturation [Nowak et al., Virology, 156, pp. 127-37 (1987)]. In some flaviviruses, the envelope glycoprotein (E) is the major virus antigen involved in virus neutralization by specific antibodies.

The complete or partial genomes of a number of WN virus isolates from the outbreak in the Northeastern United States have been sequenced. The complete sequence of WN virus isolated from a dead Chilean flamingo (WN-NY99) at the Bronx Zoo was deposited in GenBank™ (accession number AF196835) [R. S. Lanciotti et al., supra]. The genome of a WN virus isolate from human victims of the New York outbreak (WNV-NY1999) was sequenced and deposited as GenBank™ accession number AF202541 [X -Y. Jia et al., The Lancet, 354, pp. 1971-2 (1999)]. Partial sequences of isolates from two species of mosquito, a crow and a hawk from Connecticut are deposited as GenBank™ accession numbers AF206517-AF206520, respectively [J. F. Anderson et al., supra]. Comparative phylogenetic analysis of the NY sequences with previously reported WN virus sequences indicated a high degree of homology between the NY isolates and two isolates from Romania and one from Israel [J. F. Anderson et al., supra; X. -Y. Jia et al., supra; R. S. Lanciotti et al., supra].

While flaviviruses exhibit similar structural features and components, the individual viruses are significantly different at both the sequence and antigenic levels. Indeed, antigenic distinctions have been used to define four different serotypes within just the dengue virus subgroup of the flaviviruses. Infection of an individual with one dengue serotype does not provide long-term immunity against the other serotypes and secondary infections with heterologous serotypes are becoming increasingly prevalent as multiple serotypes co-circulate in a geographic area. Such secondary infections indicate that vaccination or prior infection with any one flavivirus may not to provide generalized protection against other flaviviruses. Helpful reviews on the history of attempts to develop suitable vaccines, which have especially focused on the dengue viruses, and structural features of the envelope (E) protein of flaviviruses are found in S. B. Halstead, Science, 239, pp. 476-81 (1988); W. E. Brandt, J Infect Disease, 162, pp. 577-83 (1990); T. J. Chambers et al., Ann Rev Microbiol, 44, pp. 649-88 (1990); C. W. Mandl et al., Virology, 63, pp. 564-71 (1989); and E. A. Henchal and J. R. Putnak, Clin Microbiol Rev, 3, pp. 376-96 (1990).

Despite decades of research effort, especially focusing on dengue disease, no safe and effective vaccine against any of the flaviviruses has been developed. Currently, spraying programs utilizing insecticides are the principal means for controlling the spread of WN virus. However, there are significant concerns about the toxic effects of repeated exposure to insecticides. Further, spraying does not provide complete coverage of mosquito breeding areas or eradication of mosquitoes. Accordingly, there is an urgent need for WN virus antigens for use in a vaccine and to detect the presence of a protective immune response.

### SUMMARY OF THE INVENTION

The present invention addresses the need for a vaccine to protect against WN virus by providing compositions and methods for detecting WN virus infection, conferring and detecting WN virus immunity and for preventing or reducing the spread of WN virus. Particularly, this invention provides compositions and methods comprising killed virus particles or live, infectious, attenuated viruses that are capable of eliciting the production of neutralizing or protective antibodies against WN virus. More particularly, this invention provides compositions and methods comprising purified WN virus proteins, or immunogenic fragments thereof, that are capable of eliciting the production of neutralizing or protective antibodies against WN virus. The invention further provides nucleic acid molecules encoding the polypeptides in the compositions and methods of the present invention.

In preferred embodiments, these compositions are derived from WN viral isolates from the Northeastern United States, and more particularly contain one or more WN virus E proteins or immunogenic fragments thereof. This invention further provides methods for the production and isolation of WN virus polypeptides, preferably either recombinantly or synthetically produced as described in this invention.

This invention also provides a single or multicomponent vaccine comprising one or more killed or live, infectious, attenuated WN virus particles and/or one or more WN virus polypeptides, preferably derived from WN viral isolates from the Northeastern United States and particularly the polypeptides in the compositions of the present invention. This invention also provides antibodies or antigen-binding portions thereof directed against WN virus polypeptides and immunogenic fragments and compositions and methods comprising the antibodies directed against one or more WN viral proteins or antigen-binding fragments thereof.

Also within the scope of this invention are diagnostic means and methods characterized by the pharmaceutical compositions of WN virus polypeptides or antibodies of the invention. These means and methods are useful for both the detection of WN virus infection or for the detection of a protective immune response to WN virus infection. The methods are also useful in monitoring the course of immunization against WN virus. In patients previously inoculated with the vaccines of this invention, the detection means and methods disclosed herein are also useful for determining if booster inoculations are appropriate.

### BRIEF DESCRIPTION OF THE DRAWINGS

FIG. 1 is the amino acid sequence of the WNE-121-139 fragment (SEQ ID NO: 3).

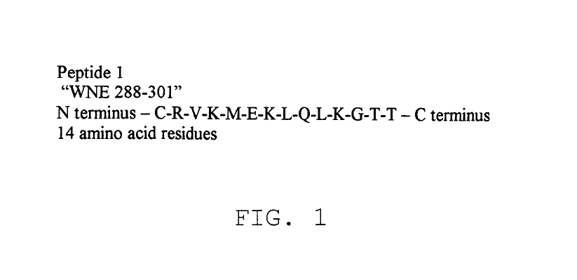

FIG. 2 is the amino acid sequence of the WNE-288-301 fragment (SEQ ID NO: 4).

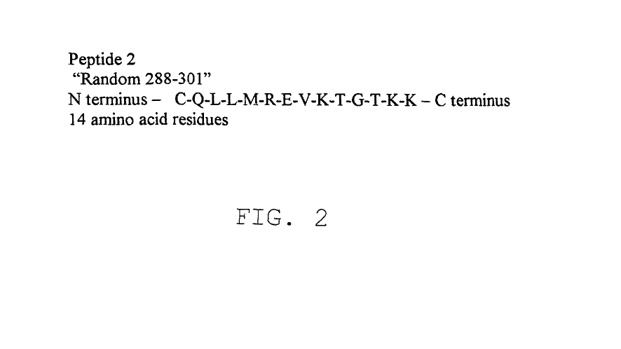

FIG. 3 is the amino acid sequence of the random-288-301 peptide (SEQ ID NO: 5).

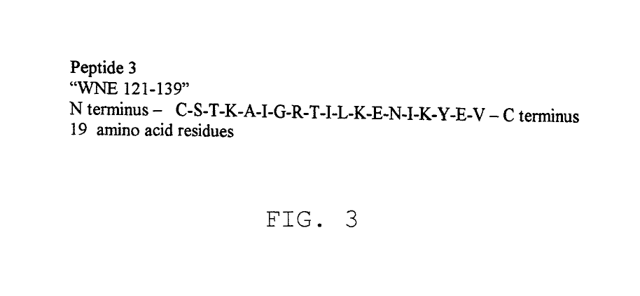

FIG. 4 is a diagrammatic representation of the 71 kDa Tr-env fusion protein. Tr, thioredoxin domain; EK, enterokinase cleavage site; WNV, 55 kDa full length sequence of West Nile virus envelope protein; V5, V5 epitopes; His, 3 kDa six histidine-tag sequence; 1, location of WNE-288-301 fragment (SEQ ID NO: 4); 2, location of WNE-121-139 fragment (SEQ ID NO: 3).

FIG. 5 is a Coomassie-blue stained SDS-PAGE gel showing purified, recombinant TR-env fusion protein.

FIG. 6 depicts the utility of mice as an experimental model organism for WN virus infection and further demonstrates that the purified Tr-env protein is able to elicit a protective antibody response. C3H mice were immunized with Tr-env protein (upper line), or Tr control protein (lower line) and challenged with West Nile virus. Five mice were in each group.

FIG. 7 shows the results of an ELISA demonstrating the specificity of antibodies generated following inoculation of mice with purified Tr-env protein. Ova, ovalbumin; Ova-random, ovalbumin-conjugated random-288-301 peptide (SEQ ID NO: 5); Ova-281, ovalcumin-conjugated WNE-288-301 peptide (SEQ ID NO: 4). 100, 1000, and 6000 represent serum dilutions of 1:100, 1:1000 and 1:6000.

FIG. 8 shows an experiment monitoring WN virus infection in mice over a range of inoculation doses.

FIG. 9 shows the results of a passive immunization experiment using antisera from C3H mice inoculated with either TR-env or TR (control).

FIG. 10 shows the results of a passive immunization experiment using antisera from C3H mice inoculated with either TR-env or TR (control).

### DETAILED DESCRIPTION OF THE INVENTION

In one aspect, this invention provides compositions and methods for detecting WN virus infection, conferring and detecting WN virus immunity and for preventing or reducing the spread of WN virus. More particularly, this invention provides compositions and methods comprising one or more killed or live, infectious, attenuated WN virus particles and/or one or more purified WN virus proteins or immunogenic fragments thereof that elicit the production of neutralizing or protective antibodies against WN virus.

The killed or live, infectious, attenuated WN virus particles in the compositions of the invention can be generated by any one of many methods know in the art. Specific examples include, but are not limited to, heat treatment to kill purified WN virus particles, passage of WN virus isolates in tissue culture to atten-uate virulence [see e.g. Dunster et al., J Gen Virol, 71, pp. 601-7 (1990)], or site-specific mutagenesis [see e.g. Mandl et al., J Virol, 74, pp. 9601-9].

As used herein, the term “polypeptide” is taken to encompass all the polypeptides, peptides, and fusion proteins described in this invention and refers to any polymer consisting essentially of amino acids regardless of its size. Although “protein” is often used in reference to relatively large polypeptides, and “peptide” is often used in reference to small polypeptides, usage of these terms in the art overlaps and varies. The term “polypeptide” as used herein thus refers interchangeably to peptides, polypeptides, or fusion proteins unless otherwise noted.

The term “amino acid” refers to a monomeric unit of a peptide, polypeptide or protein.

A “substantially pure” polypeptide is a polypeptide that is free from other WN virus components with which it is normally associated.

As used herein, a “derivative” of a WN virus polypeptide is a polypeptide in which the native form has been modified or altered. Such modifications include, but are not limited to: amino acid substitutions, modifications, additions or deletions; alterations in the pattern of lipidation, glycosylation or phosphorylation; reactions of free amino, carboxyl, or hydroxyl side groups of the amino acid residues present in the polypeptide with other organic and non-organic molecules; and other modifications, any of which may result in changes in primary, secondary or tertiary structure.

As used herein, a “protective epitope” is (1) an epitope that is recognized by a protective antibody, and/or (2) an epitope that, when used to immunize a human or animal, elicits an immune response sufficient to confer WN virus immunity or to prevent or reduce the severity for some period of time, of the resulting symptoms. A protective epitope may comprise a T cell epitope, a B cell epitope, or combinations thereof.

In preferred embodiments, this invention provides methods for the production and isolation of WN virus polypeptides, preferably either recombinantly or synthetically produced as described in this invention.

The preferred compositions and methods of the aforementioned embodiments are characterized by immunogenic polypeptides. As used herein, an “immunogenic polypeptide” is a polypeptide that, when administered to a human or animal, is capable of eliciting a corresponding antibody.

This invention also provides two novel immunogenic fragments of the WN virus E protein and compositions and methods comprising these peptides. More specifically, this invention provides the WNE-121-139 (SEQ ID NO: 3) peptide and WNE-288-301 peptide (SEQ ID NO: 4).

Also within the scope of this invention are polypeptides that are at least 75% identical in amino acid sequence to the aforementioned polypeptides. Specifically, the invention includes polypeptides that are at least 80%, 85%, 90% or 95% identical in amino acid sequence to an amino acid sequence set forth herein. The term “percent identity” in the context of amino acid sequence refers to the residues in the two sequences which are the same when aligned for maximum correspondence. There are a number of different algorithms known in the art which can be used to measure sequence similarity or identity. For instance, polypeptide sequences can be compared using NCBI BLASTp. Alternatively, Fasta, a program in GCG version 6.1. Fasta provides alignments and percent sequence identity of the regions of the best overlap between the query and search sequences (Peterson, 1990).

In another preferred embodiment, this invention provides a vaccine comprising one or more WN virus polypeptides, preferably the E protein, or one or more antibodies directed against a polypeptide present in a pharmaceutical composition of this invention.

The preferred compositions and methods of the aforementioned embodiments are characterized by WN virus polypeptides that elicit in treated humans or animals the formation of an immune reponse. As used herein, an “immune response” is manifested by the production of antibodies that recognize the corresponding polypeptide. In an especially preferred embodiment, the compositions and methods of the invention are characterized by WN virus polypeptides or antibodies that confer protection against WN virus infection or disease.

In another embodiment, this invention provides antibodies directed against a WN virus polypeptide in a pharmaceutical composition of this invention, and pharmaceutically effective compositions and methods comprising those antibodies. The antibodies of this invention are those that are specifically reactive with a polypeptide, or derivative thereof, isolated from WN virus as described in this invention. Such antibodies may be used in a variety of applications, including to detect the presence of WN virus antigens, for treatment of WN virus infection, and to confer immunity to WN virus infection.

In yet another embodiment, this invention relates to diagnostic means and methods characterized by a WN virus polypeptide or antibody of the invention.

A further embodiment of this invention provides methods for inducing immunity to WN virus in a host by administering one or more of the polypeptides, preferably derived from the WN virus E protein, or antibodies of this invention. A preferred embodiment of this invention is a method for the prevention or reduction of WN virus infection

As used herein, a “therapeutically effective amount” of a polypeptide or of an antibody is the amount that, when administered to a human or animal, elicits an immune response that is effective to confer immunity to WN virus infection or to prevent or lessen the severity, for some period of time, of a WN virus infection.

An antibody of this invention includes antibodies that specifically bind one or more of the WN virus polypeptides, preferably from an strain isolated in the Northeastern United States, as described in this invention.

As used herein, an “antibody” is an immunoglobulin molecule, or antigen-binding portion thereof, that is immunologically reactive with one or more of the purified WN virus polypeptides described in the present invention and that either was elicited by immunization with a pharmaceutical composition of this invention or was isolated or identified by its reactivity with a purified WN virus polypeptide described in the present invention.

An “antibody” refers to an intact immunoglobulin or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide.

An Fab fragment is a monovalent fragment consisting of the VL, VH, CL and CH I domains; a F(ab′)₂ fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain.

A single-chain antibody (scFv) is an antibody in which a VL and VH regions are paired to form a monovalent molecule via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444-6448, 1993, and Poljak, R. J., et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

The antibody of the invention includes human antibodies and humanized antibodies from non-human animals. The term “human antibody” includes all antibodies that have one or more variable and constant regions derived from human immunoglobulin sequences. In a preferred embodiment, all of the variable and constant domains are derived from human immunoglobulin sequences (a fully human antibody). These antibodies may be prepared in a variety of ways that are known in the art, for example, by isolation from humans infected with WN virus, by immunizing a transgenic non-human animal that produces human immunoglobulin heavy and light chains or using a recombinant combinatorial antibody library of human heavy and light chains. See, e.g., U.S. Pat. No. 6,150,584 and PCT publication number WO 94/02602, published Feb. 3, 1994.

Recombinant human antibodies of the invention in addition to the antibodies that recognize the WN virus E protein disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. There are commercially available kits for generating phage display libraries (e.g., the Pharmacia Recombinant Phage Antibody System, catalog no. 27-9400-01; and the Stratagene SurfZAP™ phage display kit, catalog no. 240612). There are also other methods and reagents that can be used in generating and screening antibody display libraries (see, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982.

A humanized antibody is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In a preferred embodiment, one or more of the CDRs are derived from a human antibody that recognizes the WN virus E protein. In a more preferred embodiment, all of the CDRs are derived from a human antibody that recognizes the WN virus E protein. In another preferred embodiment, the CDRs from multiple human antibodies that recognize the WN virus E protein are mixed and matched in a chimeric antibody. For instance, a chimeric antibody may comprise a CDR1 from the light chain of a first human antibody that recognizes the WN virus E protein may be combined with CDR2 and CDR3 from the light chain of a second human antibody that recognizes the WN virus E protein, and the CDRs from the heavy chain may be derived from a third antibody that recognizes the WN virus E protein. Further, the framework regions may be derived from one of the same antibodies, from one or more different antibodies, such as a human antibody, or from a humanized antibody.

Fragments or analogs of antibodies can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991).

The WN virus polypeptides described herein are immunologically reactive with antisera generated by immunization with the pharmaceutical compositions of the present invention or following infection with WN virus. Accordingly, they are useful in methods and compositions to detect both immunity to WN virus or prior infection with WN virus.

In addition, because at least some, if not all of the WN virus polypeptides described herein are protective proteins, they are particularly useful in single and multicomponent vaccines against WN virus infection. In this regard, multicomponent vaccines are preferred because such vaccines may be formulated to more closely resemble the immunogens presented by WN virus, and because such vaccines are more likely to confer broad-spectrum protection than a vaccine comprising only a single WN virus polypeptide.

Multicomponent vaccines according to this invention may also contain polypeptides which characterize other vaccines useful for immunization against diseases such as, for example, diphtheria, polio, hepatitis, and measles. Such multicomponent vaccines are typically incorporated into a single composition.

The preferred compositions and methods of this invention comprise WN virus polypeptides having enhanced immunogenicity. Such polypeptides may result when the native forms of the polypeptides or fragments thereof are modified or subjected to treatments to enhance their immunogenic character in the intended recipient.

Numerous techniques are available and well known to those of skill in the art which may be used, without undue experimentation, to substantially increase the immunogenicity of the WN virus polypeptides described herein. For example, a WN virus polypeptide used in a pharmaceutical composition or vaccine of this invention may be modified by coupling to dinitrophenol groups or arsanilic acid, or by denaturation with heat and/or SDS. Particularly if the polypeptides are small, chemically synthesized polypeptides, it may be desirable to couple them to an immunogenic carrier. The coupling, of course, must not interfere with the ability of either the polypeptide or the carrier to function appropriately. For a review of some general considerations in coupling strategies, see Antibodies, A Laboratory Manual, Cold Spring Harbor Laboratory, ed. E. Harlow and D. Lane (1988).

Useful immunogenic carriers are well known in the art. Examples of such carriers are keyhole limpet hemocyanin (KLH); albumins such as bovine serum albumin (BSA) and ovalbumin, PPD (purified protein derivative of tuberculin); red blood cells; tetanus toxoid; cholera toxoid; agarose beads; activated carbon; or bentonite.

Modification of the amino acid sequence of the polypeptides disclosed herein to generate derivatives with altered lipidation states is also a method which may be used to increase their immunogenicity or alter their biochemical properties. For example, the polypeptides or fragments thereof may be expressed with or without the signal and other sequences that may direct addition of lipid moieties.

As will be apparent from the disclosure to follow, the polypeptides in the pharmaceutical compositions of this invention may also be prepared with the objective of increasing stability or rendering the molecules more amenable to purification and preparation. one such technique is to express the polypeptides as fusion proteins comprising other WN virus sequences.

In accordance with this invention, a derivative of a polypeptide of the invention may be prepared by a variety of methods, including by in vitro manipulation of the DNA encoding the native polypeptides and subsequent expression of the modified DNA, by chemical synthesis of derivatized DNA sequences, or by chemical or biological manipulation of expressed amino acid sequences.

For example, derivatives may be produced by substitution of one or more amino acids with a different natural amino acid, an amino acid derivative or non- native amino acid. Those of skill in the art will understand that conservative substitution is preferred, e.g., 3-methyl-histidine may be substituted for histidine, 4-hydroxy-proline may be substituted for proline, 5-hydroxylysine may be substituted for lysine, and the like.

Furthermore, one of skill in the art will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W. H. Freeman and Co.

Conservative substitutions typically include the substitution of one amino acid for another with similar characteristics such as substitutions within the following groups: valine, glycine; glycine, alanine; valine, isoleucine; aspartic acid, glutamic acid; asparagine, glutamine; serine, threonine; lysine, arginine; and phenylalanine, tyrosine. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

Other conservative substitutions can be taken from Table 1, and yet others are described by Dayhoff in the Atlas of Protein Sequence and Structure (1988).

Causing amino acid substitutions which are less conservative may also result in desired derivatives, e.g., by causing changes in charge, conformation or other biological properties. Such substitutions would include for example, substitution of a hydrophilic residue for a hydrophobic residue, substitution of a cysteine or proline for another residue, substitution of a residue having a small side chain for a residue having a bulky side chain or substitution of a residue having a net positive charge for a residue having a net negative charge.

When the result of a given substitution cannot be predicted with certainty, the derivatives may be readily assayed according to the methods disclosed herein to determine the presence or absence of the desired characteristics. In particular, the immunogenicity, immunodominance and/or protectiveness of a derivative polypeptide used in a pharmaceutical composition of this invention can be readily determined using methods disclosed in the Examples.

In a preferred embodiment of this invention, the WN virus polypeptides described herein are prepared as part of a larger fusion protein. For example, a WN virus polypeptide used in a pharmaceutical composition of this invention may be fused at its N-terminus or C-terminus to a different immunogenic WN virus polypeptide, to a non-WN virus polypeptide or to combinations thereof, to produce fusion proteins comprising the WN virus polypeptide.

In a preferred embodiment of this invention, fusion proteins comprising a WN virus polypeptide used in a pharmaceutical composition are constructed comprising B cell and/or T cell epitopes from multiple strains of WN virus, each variant differing from another with respect to the locations or sequences of the epitopes within the polypeptide. Such fusion proteins are particularly effective in the induction of immunity against a wide spectrum of WN virus strains.

In another preferred embodiment of this invention, the WN virus polypeptides used in pharmaceutical compositions are fused to moieties, such as immunoglobulin domains, which may increase the stability and prolong the in vivo plasma half-life of the polypeptide. Such fusions may be prepared without undue experimentation according to methods well known to those of skill in the art, for example, in accordance with the teachings of U.S. Pat. No. 4,946,778, or U.S. Pat. No. 5,116,964. The exact site of the fusion is not critical as long as the polypeptide retains the desired biological activity. Such determinations may be made according to the teachings herein or by other methods known to those of skill in the art.

It is preferred that the fusion proteins comprising the WN virus polypeptides be produced at the DNA level, e.g., by constructing a nucleic acid molecule encoding the fusion protein, transforming host cells with the mol-ecule, inducing the cells to express the fusion protein, and recovering the fusion protein from the cell culture. Alternatively, the fusion proteins may be produced after gene expression according to known methods.

The polypeptides of the invention may also be part of larger multimeric molecules which may be produced recombinantly or may be synthesized chemically. Such multimers may also include the polypeptides fused or coupled to moieties other than amino acids, including lipids and carbohydrates.

Preferably, the multimeric proteins will consist of multiple T or B cell epitopes or combinations thereof repeated within the same molecule, either randomly, or with spacers (amino acid or otherwise) between them.

In a preferred embodiment of this invention, antigens from WN virus strains isolated in the Northeastern United States are incorporated into a vaccine.

In another embodiment of this invention, a WN virus polypeptide used in a pharmaceutical composition of this invention, preferably a WN virus polypeptide which is also a protective WN virus polypeptide, is incorporated into a single component vaccine. In another embodiment of this invention, WN virus polypeptides which are also protective polypeptides are incorporated into a multicomponent vaccine comprising other protective polypeptides. In addition, a multicomponent vaccine may also contain protective polypeptides useful for immunization against other diseases such as, for example, diphtheria, polio, hepatitis, and measles. Such a vaccine, by virtue of its ability to elicit antibodies to a variety of protective WN virus polypeptides, will be effective to protect against WN virus infection by a broad spectrum of WN virus strains, even those that may not express a variant of one or more of the WN virus proteins that is cross-reactive with the polypeptides of one particular strain.

The multicomponent vaccine may contain a WN virus polypeptide as part of a multimeric molecule in which the various components are covalently associated. Alternatively, it may contain multiple individual components. For example, a multicomponent vaccine may be prepared comprising two or more of the WN virus polypeptides, wherein each polypeptide is expressed and purified from independent cell cultures and the polypeptides are combined prior to or during formulation.

Alternatively, a multicomponent vaccine may be prepared from heterodimers or tetramers wherein the polypeptides have been fused to immunoglobulin chains or portions thereof. Such a vaccine could comprise, for example, a WNE-121-139 polypeptide (SEQ ID NO: 3) fused to an immunoglobulin heavy chain and a WNE-288-301 polypeptide (SEQ ID NO: 4), fused to an immunoglobulin light chain, and could be produced by transforming a host cell with DNA encoding the heavy chain fusion and DNA encoding the light chain fusion. One of skill in the art will understand that the host cell selected should be capable of assembling the two chains appropriately. Alternatively, the heavy and light chain fusions could be produced from separate cell lines and allowed to associate after purification.

The desirability of including a particular component and the relative proportions of each component may be determined by using the assay systems disclosed herein, or by using other systems known to those in the art. Most preferably, the multicomponent vaccine will comprise numerous T cell and B cell epitopes of protective WN virus polypeptides.

This invention also contemplates that a WN virus polypeptide described in this invention, either alone or combined, may be administered to an animal via a liposome delivery system in order to enhance their stability and/or immunogenicity. Delivery of a WN virus polypeptide via liposomes may be particularly advantageous because the liposome may be internalized by phagocytic cells in the treated animal. Such cells, upon ingesting the liposome, would digest the liposomal membrane and subsequently present the polypeptide to the immune system in conjunction with other molecules required to elicit a strong immune response.

The liposome system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 4,762,915, 5,000,958, 5,169,637 or 5,185,154. In addition, it may be desirable to express the WN virus polypeptides described in this invention as lipoproteins, in order to enhance their binding to liposomes.

Any of the polypeptides used in a pharmaceutical composition of this invention may be used in the form of a pharmaceutically acceptable salt. Suitable acids and bases which are capable of forming salts with the polypeptides of the present invention are well known to those of skill in the art, and include inorganic and organic acids and bases.

According to this invention, we describe a method which comprises the steps of treating a subject, including a human, with a therapeutically effective amount of a WN virus polypeptide, preferably from a WN virus strain isolated in the Northeastern United States, or a fusion protein or a multimeric protein comprising a WN virus polypeptide, in a manner sufficient to confer immunity to WN virus infection or prevent or reduce the severity, for some period of time, of the symptoms of WN virus infection. The polypeptides that are preferred for use in such methods are those that contain protective epitopes. Such protective epitopes may be B cell epitopes, T cell epitopes, or combinations thereof.

According to another embodiment of this invention, we describe a method which comprises the steps of treating a subject, including a human, with a multicomponent vaccine comprising a therapeutically effective amount of a WN virus polypeptide, or a fusion protein or multimeric protein comprising such polypeptide in a manner sufficient to confer immunity to WN virus infection, or prevent or reduce the severity, for some period of time, of the symptoms of WN virus infection. Again, the polypeptides, fusion proteins and multimeric proteins that are preferred for use in such methods are those that contain protective epitopes, which may be B cell epitopes, T cell epitopes, or combinations thereof.

The most preferred polypeptides, fusion proteins and multimeric proteins for use in these compositions and methods are those containing both strong T cell and B cell epitopes. Without being bound by theory, we believe that this is the best way to stimulate high titer anti-bodies that are effective to confer immunity to WN virus infection. Such preferred polypeptides will be internalized by B cells expressing surface immunoglobulin that recognizes the B cell epitope(s). The B cells will then process the antigen and present it to T cells. The T cells will recognize the T cell epitope(s) and respond by proliferating and producing lymphokines which in turn cause B cells to differentiate into antibody producing plasma cells. Thus, in this system, a closed autocatalytic circuit exists which will result in the amplification of both B and T cell responses, leading ultimately to production of a strong immune response which includes high titer antibodies against the WN virus polypeptide.

One of skill in the art will also understand that it may be advantageous to administer a pharmaceutical composition containing WN virus polypeptide as described in this invention in a form that will favor the production of T-helper cells type 1 (T_{H}1), which help activate macrophages, and/or T-helper cells type 2 (T_{H}2), which help B cells to generate antibody responses. Aside from administering epitopes which are strong T cell or B cell epitopes, the induction of T_{H}1 or T_{H}2 cells may also be favored by the mode of administration of the polypeptide. For example, a WN virus polypeptide may be administered in certain doses or with particular adjuvants and immunomodulators, for example with interferon-gamma or interleukin-12 (T_{H}1 response) or interleukin-4 or interleukin-10 (T_{H}2 response).

To prepare the preferred polypeptides for use in a pharmaceutical composition of this invention, in one embodiment, overlapping fragments of WN virus polypeptides are constructed. The polypeptides that contain B cell epitopes may be identified in a variety of ways for example by their ability to (1) remove protective antibodies from polyclonal antiserum directed against the polypeptide or (2) elicit an immune response which is effective to confer immunity to WN virus infection, or prevent or reduce the severity, for some period of time, of the symptoms of WN virus infection.

Alternatively, the polypeptides may be used to produce monoclonal antibodies which are screened for their ability to confer immunity to WN virus infection, or prevent or reduce the severity, for some period of time, of the symptoms of WN virus infection, when used to immunize naive animals. Once a given monoclonal antibody is found to confer protection, the particular epitope that is recognized by that antibody may then be identified.

As recognition of T cell epitopes is MHC restricted, the polypeptides that contain T cell epitopes may be identified in vitro by testing them for their ability to stimulate proliferation and/or cytokine production by T cell clones generated from humans of various HLA types, from the lymph nodes, spleens, or peripheral blood lymphocytes of C3H or other laboratory mice, or from domestic animals.

In a preferred embodiment of the present invention, a WN virus polypeptide containing a B cell epitope is fused to one or more other immunogenic WN virus polypeptides containing strong T cell epitopes. The fusion protein that carries both strong T cell and B cell epitopes is able to participate in elicitation of a high titer antibody response effective to confer immunity to WN virus infection.

Strong T cell epitopes may also be provided by non-WN virus molecules. For example, strong T cell epitopes have been observed in hepatitis B virus core antigen (HBcAg). Furthermore, it has been shown that linkage of one of these segments to segments of the surface antigen of Hepatitis B virus, which are poorly recognized by T cells, results in a major amplification of the anti-HBV surface antigen response, [D. R. Milich et al., Nature, 329, pp. 547-49 (1987)].

Therefore, in yet another preferred embodiment, B cell epitopes of the WN virus polypeptides are fused to segments of HBcAG or to other antigens which contain strong T cell epitopes, to produce a fusion protein that can elicit a high titer antibody response against WN virus antigens. In addition, it may be particularly advantageous to link an WN virus polypeptide for use in a pharmaceutical composition of this invention to a strong immunogen that is also widely recognized, for example tetanus toxoid.

It will be readily appreciated by one of ordinary skill in the art that the polypeptides in the pharmaceutical compositions of this invention, as well as fusion proteins and multimeric proteins containing them, may be prepared by recombinant means, chemical means, or combinations thereof.

For example, the polypeptides may be generated by recombinant means using the DNA sequence as set forth in the sequence listing contained herein. DNA encoding variants of the polypeptides in other WN virus strains may likewise be cloned, e.g., using PCR and oligonucleotide primers derived from the sequence herein disclosed.

In this regard, it may be particularly desirable to isolate the genes encoding WN virus polypeptides from any isolates that may differ antigenically, i.e., WN virus isolates against which the pharmaceutical compositions described in the present invention which are initially used for vaccine development are ineffective to protect, in order to obtain a broad spectrum of different epitopes which would be useful in the methods and compositions of this invention.

Oligonucleotide primers and other nucleic acid probes derived from the genes encoding the polypeptides in the pharmaceutical compositions of this invention may also be used to isolate and clone related proteins from other WN virus isolates which may contain regions of DNA sequence homologous to the DNA sequences of the polypeptides described in this invention.

In a preferred embodiment, the polypeptides used in the pharmaceutical compositions of this invention are produced recombinantly and may be expressed in unicellular hosts. As is well known to one of skill in the art, in order to obtain high expression levels of foreign DNA sequences in a host, the sequences are generally operably linked to transcriptional and translational expression control sequences that are functional in the chosen host. Preferably, the expression control sequences, and the gene of interest, will be contained in an expression vector that further comprises a selection marker.

The DNA sequences encoding the polypeptides used in the pharmaceutical compositions of this invention may or may not encode a signal sequence. If the expression host is eukaryotic, it generally is preferred that a signal sequence be encoded so that the mature protein is secreted from the eukaryotic host.

An amino terminal methionine may or may not be present on the expressed polypeptides in the pharmaceutical compositions of this invention. If the terminal methionine is not cleaved by the expression host, it may, if desired, be chemically removed by standard techniques.

A wide variety of expression host/vector combinations may be employed in expressing the DNA sequences encoding the WN virus polypeptides used in the pharmaceutical compositions and vaccines of this invention. Useful expression vectors for eukaryotic hosts, include, for example, vectors comprising expression control sequences from SV40, bovine papilloma virus, adenovirus, adeno-associated virus, cytomegalovirus and retroviruses including lentiviruses. Useful expression vectors for bacterial hosts include bacterial plasmids, such as those from E. coli, including pBluescript®, pGEX-2T, pUC vectors, col E1, pCR1, pBR322, pMB9 and their derivatives, pET-15, wider host range plasmids, such as RP4, phage DNAs, e.g., the numerous derivatives of phage lambda, e.g. λGT10 and λGT11, and other phages. Useful expression vectors for yeast cells include the 2 μ plasmid and derivatives thereof. Useful vectors for insect cells include pVL 941.

In addition, any of a wide variety of expression control sequences—sequences that control the expression of a DNA sequence when operably linked to it—may be used in these vectors to express the polypeptides used in the pharmaceutical compositions of this invention. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the T3 and T7 promoters, the major operator and promoter regions of phage lambda, the control regions of fd coat protein, the promoter for 3-phosphoglycerate kinase or other glycolytic enzymes, the promoters of acid phosphatase, e.g., Pho5, the promoters of the yeast α-mating system and other constitutive and inducible promoter sequences known to control the expression of genes of prokaryotic or eukaryotic cells or their viruses, and various combinations thereof.

In a preferred embodiment, a DNA sequence encoding a WN virus polypeptide used in a pharmaceutical composition of this invention is cloned in the expression vector lambda ZAP® II (Stratagene, La Jolla, Calif.), in which expression from the lac promoter may be induced by IPTG.

In another preferred embodiment, a DNA sequence encoding a WN virus polypeptide, preferably the E protein, that is used in a pharmaceutical composition of this invention is cloned in the pBAD/Thiofusion™ expression vector, in which expression of the resulting thioredoxin fusion protein from the araBAD promoter may be induced by arabinose.

In yet another preferred embodiment, DNA encoding the WN virus polypeptides used in a pharmaceutical composition of this invention is inserted in frame into an expression vector that allows high level expression of the polypeptide as a glutathione S-transferase fusion protein. Such a fusion protein thus contains amino acids encoded by the vector sequences as well as amino acids of the WN virus polypeptide.

The term “host cell” refers to one or more cells into which a recombinant DNA molecule is introduced. Host cells of the invention include, but need not be limited to, bacterial, yeast, animal and plant cells. Host cells can be unicellular, or can be grown in tissue culture as liquid cultures, monolayers or the like. Host cells may also be derived directly or indirectly from tissues.

A wide variety of unicellular host cells are useful in expressing the DNA sequences encoding the polypeptides used in the pharmaceutical compositions of this invention. These hosts may include well known eukaryotic and prokaryotic hosts, such as strains of E. coli, Pseudomonas, Bacillus, Streptomyces, fungi, yeast, insect cells such as Spodoptera frugiperda (SF9), animal cells such as CHO and mouse cells, African green monkey cells such as COS 1, COS 7, BSC 1, BSC 40, and BMT 10, and human cells, as well as plant cells.

A host cell is “transformed” by a nucleic acid when the nucleic acid is translocated into the cell from the extracellular environment. Any method of transferring a nucleic acid into the cell may be used; the term, unless otherwise indicated herein, does not imply any particular method of delivering a nucleic acid into a cell, nor that any particular cell type is the subject of transfer.

An “expression control sequence” is a nucleic acid sequence which regulates gene expression (i.e., transcription, RNA formation and/or translation). Expression control sequences may vary depending, for example, on the chosen host cell or organism (e.g., between prokaryotic and eukaryotic hosts), the type of transcription unit (e.g., which RNA polymerase must recognize the sequences), the cell type in which the gene is normally expressed (and, in turn, the biological factors normally present in that cell type).

A “promoter” is one such expression control sequence, and, as used herein, refers to an array of nucleic acid sequences which control, regulate and/or direct transcription of downstream (3′) nucleic acid sequences. As used herein, a promoter includes necessary nucleic acid sequences near the start site of transcription, such as, in the case of a polymerase II type promoter, a TATA element.

A “constitutive” promoter is a promoter which is active under most environmental and developmental conditions. An “inducible” promoter is a promoter which is inactive under at least one environmental or developmental condition and which can be switched “on” by altering that condition. A “tissue specific” promoter is active in certain tissue types of an organism, but not in other tissue types from the same organism. Similarly, a developmentally-regulated promoter is active during some but not all developmental stages of a host organism.

Expression control sequences also include distal enhancer or repressor elements which can be located as much as several thousand base pairs from the start site of transcription. They also include sequences required for RNA formation (e.g., capping, splicing, 3′ end formation and poly-adenylation, where appropriate); translation (e.g., ribosome binding site); and post-translational modifications (e.g., glycosylation, phosphorylation, methylation, prenylation, and the like).

The term “operably linked” refers to functional linkage between a nucleic acid expression control sequence (such as a promoter, or array of transcription factor binding sites) and a second nucleic acid sequence, wherein the expression control sequence directs transcription of the nucleic acid corresponding to the second sequence.

It should of course be understood that not all vectors and expression control sequences will function equally well to express the WN virus polypeptides mentioned herein. Neither will all hosts function equally well with the same expression system. However, one of skill in the art may make a selection among these vectors, expression control sequences and hosts without undue experimentation and without departing from the scope of this invention. For example, in selecting a vector, the host must be considered because the vector must be replicated in it. The vector's copy number, the ability to control that copy number, the ability to control integration, if any, and the expression of any other proteins encoded by the vector, such as antibiotic or other selection markers, should also be considered.

In selecting an expression control sequence, a variety of factors should also be considered. These include, for example, the relative strength of the promoter sequence, its controllability, and its compatibility with the DNA sequence of the peptides described in this invention, particularly with regard to potential secondary structures. Unicellular hosts should be selected by consideration of their compatibility with the chosen vector, the toxicity of the product coded for by the DNA sequences encoding the proteins used in a pharmaceutical composition of this invention, their secretion characteristics, their ability to fold the polypeptide correctly, their fermentation or culture requirements, and the ease of purification from them of the products coded for by the DNA sequences.

Within these parameters, one of skill in the art may select various vector/expression control sequence/host combinations that will express the DNA sequences encoding the products used in the pharmaceutical compositions of this invention on fermentation or in other large scale cultures.

The polypeptides described in this invention may be isolated from the fermentation or cell culture and purified using any of a variety of conventional methods including: liquid chromatography such as normal or reversed phase, using HPLC, FPLC and the like; affinity chromatography (such as with inorganic ligands or monoclonal antibodies); size exclusion chromatography; immobilized metal chelate chromatography; gel electrophoresis; and the like. One of skill in the art may select the most appropriate isolation and purification techniques without departing from the scope of this invention. If the polypeptide is membrane bound or suspected of being a lipoprotein, it may be isolated using methods known in the art for such proteins, e.g., using any of a variety of suitable detergents.

In addition, the polypeptides of the invention may be generated by any of several chemical techniques. For example, they may be prepared using the solid-phase synthetic technique originally described by R. B. Merrifield, J Am Chem Soc, 83, pp. 2149-54 (1963), or they may be prepared by synthesis in solution. A summary of peptide synthesis techniques may be found in E. Gross & H. J. Meinhofer, 4 The Peptides: Analysis, Synthesis, Biology; Modern Techniques Of Peptide And Amino Acid Analysis, John Wiley & Sons, (1981) and M. Bodanszky, Principles Of Peptide Synthesis, Springer-Verlag (1984).

Typically, these synthetic methods comprise the sequential addition of one or more amino acid residues to a growing peptide chain. Often peptide coupling agents are used to facilitate this reaction. For a recitation of peptide coupling agents suitable for the uses described herein see M. Bodansky, supra. Normally, either the amino or carboxyl group of the first amino acid residue is protected by a suitable, selectively removable protecting group. A different protecting group is utilized for amino acids containing a reactive side group, e.g., lysine. A variety of protecting groups known in the field of peptide synthesis and recognized by conventional abbreviations therein, may be found in T. Greene, Protective Groups In Organic Synthesis, Academic Press (1981).

According to another embodiment of this invention, antibodies directed against a WN virus polypeptide are generated. Such antibodies are immunoglobulin molecules or portions thereof that are immunologically reactive with a polypeptide of the present invention. It should be understood that the antibodies of this invention include antibodies immunologically reactive with fusion proteins and multimeric proteins comprising a WN virus polypeptide.

Antibodies directed against a WN virus polypeptide may be generated by a variety of means including immunizing a mammalian host with WN virus extract or infection with WN virus, or by immunization of a mammalian host with a WN virus polypeptide of the present invention. Such antibodies may be polyclonal or monoclonal; it is preferred that they are monoclonal. Methods to produce polyclonal and monoclonal antibodies are well known to those of skill in the art. For a review of such methods, see Antibodies, A Laboratory Manual, supra, and D. E. Yelton, et al., Ann Rev Biochem, 50, pp. 657-80 (1981). Determination of immunoreactivity with a WN virus polypeptide used in a pharmaceutical composition of this invention may be made by any of several methods well known in the art, including by immunoblot assay and ELISA.

An antibody of this invention may also be a hybrid molecule formed from immunoglobulin sequences from different species (e.g., mouse and human ) or from portions of immunoglobulin light and heavy chain sequences from the same species. It may be a molecule that has multiple binding specificities, such as a bifunctional antibody prepared by any one of a number of techniques known to those of skill in the art including: the production of hybrid hybridomas; disulfide exchange; chemical cross-linking; addition of peptide linkers between two monoclonal antibodies; the introduction of two sets of immunoglobulin heavy and light chains into a particular cell line; and so forth.

The antibodies of this invention may also be human monoclonal antibodies produced by any of the several methods known in the art. For example, human monoclonal antibodies may be produced by immortalized human cells, by SCID-hu mice or other non-human animals capable of producing “human” antibodies, by the expression of cloned human immunoglobulin genes, by phage-display, or by any other method known in the art.

In addition, it may be advantageous to couple the antibodies of this invention to toxins such as diphtheria, pseudomonas exotoxin, ricin A chain, gelonin, etc., or antibiotics such as penicillins, tetracyclines and chloramphenicol.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

One of skill in the art will understand that antibodies directed against a pharmaceutical composition of the invention may have utility in prophylactic compositions and methods directed against WN virus infection. For example, the level of WN virus in infected mosquitoes may be decreased by allowing them to feed on the blood of animals immunized with a pharmaceutical composition or vaccine of this invention.

The antibodies of this invention also have a variety of other uses. For example, they are useful as reagents to screen for expression of the WN virus polypeptides, either in libraries constructed from WN virus DNA or from other samples in which the proteins may be present. Moreover, by virtue of their specific binding affinities, the antibodies of this invention are also useful to purify or remove polypeptides from a given sample, to block or bind to specific epitopes on the polypeptides and to direct various molecules, such as toxins, to mosquitoes serving as vectors for WN virus.

To screen the pharmaceutical compositions, vaccines and antibodies of this invention for their ability to confer protection against WN virus infection or their ability to reduce the severity or duration of the attendant symptoms, mice are preferred as an animal model. Of course, while any animal that is susceptible to WN virus infection may be useful, mice are a well-known and particularly convenient model. Thus, by administering a particular WN virus polypeptide or anti-WN virus polypeptide antibody to mice, one of skill in the art may determine without undue experimentation whether that polypeptide or antibody would be useful in the methods and compositions claimed herein.

The administration of the WN virus polypeptide or antibody of this invention to the animal may be accomplished by any of the methods disclosed herein or by a variety of other standard procedures. For a detailed discussion of such techniques, see Antibodies, A Laboratory Manual, supra. Preferably, if a polypeptide is used, it will be administered with a pharmaceutically acceptable adjuvant, such as complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

Once the pharmaceutical compositions, vaccines or antibodies of this invention have been determined to be effective in the screening process, they may then be used in a therapeutically effective amount in pharmaceutical compositions and methods to confer immunity to WN virus infection in humans and animals and to prevent or reduce the transmission of WN virus from non-human host animals.

The pharmaceutical compositions of this invention may be in a variety of conventional depot forms. These include, for example, solid, semi-solid and liquid dosage forms, such as tablets, pills, powders, liquid solutions or suspensions, liposomes, capsules, suppositories, injectable and infusible solutions. The preferred form depends upon the intended mode of administration and prophylactic application.

Such dosage forms may include pharmaceutically acceptable carriers and adjuvants which are known to those of skill in the art. These carriers and adjuvants include, for example, RIBI, ISCOM, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes such as protamine sulfate, disodium hydrogen phosphate, sodium chloride, zinc salts, colloidal silica, magnesium trisilicate, polyvinyl pyrrolidone, cellulose-based substances, and polyethylene glycol. Adjuvants for topical or gel base forms may be selected from the group consisting of sodium carboxymethylcellulose, polyacrylates, polyoxyethylene-polyoxypropylene-block polymers, polyethylene glycol, and wood wax alcohols.

The vaccines and compositions of this invention may also include other components or be subject to other treatments during preparation to enhance their immunogenic character or to improve their tolerance in patients.

Compositions comprising an antibody of this invention may be administered by a variety of dosage forms and regimens similar to those used for other passive immunotherapies and well known to those of skill in the art. Generally, the WN virus polypeptides may be formulated and administered to the patient using methods and compositions similar to those employed for other pharmaceutically important polypeptides (e.g., the vaccine against hepatitis).

Any pharmaceutically acceptable dosage route, including parenteral, intravenous, intramuscular, intralesional or subcutaneous injection, may be used to administer the polypeptide or antibody composition. For example, the composition may be administered to the patient in any pharmaceutically acceptable dosage form including those which may be administered to a patient intravenously as bolus or by continued infusion over a period of hours, days, weeks or months, intramuscularly—including paravertebrally and periarticularly—subcutaneously, intracutaneously, intra-articularly, intrasynovially, intrathecally, intralesionally, periostally or by oral or topical routes. Preferably, the compositions of the invention are in the form of a unit dose and will usually be administered to the patient intramuscularly.

The pharmaceutical compositions, vaccines or antibodies of this invention may be administered to the patient at one time or over a series of treatments. The most effective mode of administration and dosage regimen will depend upon the level of immunogenicity, the particular composition and/or adjuvant used for treatment, the severity and course of the expected infection, previous therapy, the patient's health status and response to immunization, and the judgment of the treating physician.

For example, in an immunocompetent patient, the more highly immunogenic the polypeptide, the lower the dosage and necessary number of immunizations. Similarly, the dosage and necessary treatment time will be lowered if the polypeptide is administered with an adjuvant. Generally, the dosage will consist of 10 μg to 100 mg of the purified polypeptide, and preferably, the dosage will consist of 10-1000 μg. Generally, the dosage for an antibody will be 0.5 mg-3.0 g.

In a preferred embodiment of this invention, the WN virus polypeptide is administered with an adjuvant, in order to increase its immunogenicity. Useful adjuvants include RIBI, and ISCOM, simple metal salts such as aluminum hydroxide, and oil based adjuvants such as complete and incomplete Freund's adjuvant. When an oil based adjuvant is used, the polypeptide usually is administered in an emulsion with the adjuvant.

In yet another preferred embodiment, E. coli expressing proteins comprising an WN virus polypeptide are administered orally to non-human animals according to methods known in the art, to confer immunity to WN virus infection and to prevent or reduce the transmission of WN virus from non-human animals. For example, a palatable regimen of bacteria expressing a WN virus polypeptide, alone or in the form of a fusion protein or multimeric protein, may be administered with animal food to be consumed by wild birds or other animals that act as alternative hosts for WN virus.

Ingestion of such bacteria may induce an immune response comprising both humoral and cell-mediated components. See J. C. Sadoff et al., Science, 240, pp. 336-38 (1988); K. S. Kim et al., Inf Immun, 57, pp. 2434-40 (1989); M. Dunne et al., Inf Immun, 63, pp. 1611-4 (1995); E. Fikrig et al., J Infec Dis, 164, 1224-7 (1991).

Moreover, the level of pathogens in mosquitoes feeding on such animals may be lessened or eliminated, thus inhibiting transmission to the next animal.

According to yet another embodiment, the WN virus polypeptides used in the pharmaceutical compositions of this invention, preferably, are useful as diagnostic agents for detecting immunity to WN virus or prior infection with WN virus. The polypeptides are capable of binding to antibody molecules produced in animals, including humans, that have been exposed to WN virus antigens as a result of infection with WN virus or vaccination with a pharmaceutical composition of this invention. The detection of WN virus antigens is evidence of prior exposure to WN virus. Such information is an important aid in the diagnosis of WN virus infection.

Such diagnostic agents may be included in a kit which may also comprise instructions for use and other appropriate reagents, preferably a means for detecting when the polypeptide or antibody is bound. For example, the polypeptide or antibody may be labeled with a detection means that allows for the detection of the polypeptide when it is bound to an antibody, or for the detection of the antibody when it is bound to WN virus or an antigen thereof.

The detection means may be a fluorescent labeling agent such as fluorescein isocyanate (FIC), fluorescein isothiocyanate (FITC), and the like, an enzyme, such as horseradish peroxidase (HRP), glucose oxidase or the like, a radioactive element such as ¹²⁵I or ⁵¹Cr that produces gamma ray emissions, or a radioactive element that emits positrons which produce gamma rays upon encounters with electrons present in the test solution, such as ¹¹C, ¹⁵O, or ¹³N. Binding may also be detected by other methods, for example via avidin-biotin complexes.

The linking of the detection means is well known in the art. For instance, monoclonal antibody molecules produced by a hybridoma can be metabolically labeled by incorporation of radioisotope-containing amino acids in the culture medium, or polypeptides may be conjugated or coupled to a detection means through activated functional groups.

The diagnostic kits of the present invention may be used to detect the presence of anti-WN virus antibodies in a body fluid sample such as serum, plasma or urine. Thus, in preferred embodiments, a WN virus polypeptide or an antibody of the present invention is bound to a solid support typically by adsorption from an aqueous medium. Useful solid matrices are well known in the art, and include crosslinked dextran; agarose; polystyrene; polyvinylchloride; cross-linked polyacrylamide; nitrocellulose or nylon-based materials; tubes, plates or the wells of microtiter plates. The polypeptides or antibodies of the present invention may be used as diagnostic agents in solution form or as a substantially dry powder, e.g., in lyophilized form.

WN virus polypeptides and antibodies directed against those polypeptides provide much more specific diagnostic reagents than whole WN virus and thus may alleviate such pitfalls as false positive and false negative results.

One skilled in the art will realize that it may also be advantageous in the preparation of detection reagents to utilize epitopes from more than one WN virus protein or more than one WN virus isolate and antibodies directed against such epitopes.

The skilled artisan also will realize that it may be advantageous to prepare a diagnostic kit comprising diagnostic reagents to detect WN virus as well as pathogens found in the same mosquito vector, for example other flaviviruses which are known to exhibit similar symptoms, and instructions for their use.

The compositions and methods comprising the polypeptides and antibodies of the present invention may also be useful for prevention of infection by other strains of WN virus which may express proteins sharing amino acid sequence or conformational similarities with the WN virus polypeptides of the present invention.

Throughout this specification and claims, the word “comprise,” or variations such as “comprises” or “comprising,” will be understood to imply the inclusion of a stated integer or group of integers but not the exclusion of any other integer or group of integers.

In order that this invention may be better understood, the following examples are set forth. These examples are for purposes of illustration only, and are not to be construed as limiting the scope of the invention in any manner.

### EXAMPLE I

### Isolation of WN Virus in Connecticut

We obtained several WN virus isolates from mosquitoes and birds in Connecticut. Mosquitoes were captured in dry ice-baited Centers for Disease Control miniature light traps. One mosquito trap was placed at each location per night; the numbers of traps per site ranged from 1 to 6. Mosquitoes were transported alive to the laboratory where they were identified and grouped (pooled) according to species, collecting site, and date. The number of mosquitoes per pool ranged from 1 to 50. The total number of mosquitoes by species that were collected in 14 towns in Fairfield County, Conn., and tested for virus from Sep. 6 through Oct. 14, 1999: Aedes vexans, 1688; Ae. cinereus, 172; Ae. trivittatus, 131; Ae. taeniorhynchus, 123; Ae. sollicitans, 109; Ae. cantator, 63; Ae. triseriatus, 28; Ae. japonicus, 19; Ae. canadensis, 1; Anopheles punctipennis, 82; An. quadrimaculatus, 4; An. walkeri, 2; Coquillettidia perturbans, 15; Culex pipiens, 744; Cx. restuans, 27; Cx. erraticus, 4; Cx. territans, 1; Culiseta melanura, 76; Cs. morsitans, 1; Psorophora ferox, 4; and Uranotaenia sapphirina, 104. Mosquitoes were stored at −80° C. until tested for virus. Additionally, we obtained isolated West Nile virus from mosquitoes collected in New York City.

Most dead birds were collected by state or town personnel in Connecticut and sent to the Pathobiology Department at the University of Connecticut, Storrs, where they were examined for postmortem and nutritional condition, gross lesions, and microscopic evidence indicative of encephalitis. Brain tissue from birds with presumed encephalitis was frozen at −70° C. and then sent to the Connecticut Agricultural Experiment Station, New Haven, for virus testing. Connecticut towns from which dead crows were collected and virus isolated from brain tissues (number of isolates in parentheses): Bridgeport (1), Darien (1), Fairfield (4), Greenwich (3), Hamden (1), Madison (1), Milford (1), New Canaan (1), New Haven (3), North Haven (1), Norwalk (1), Redding (1), Stamford (5), Stratford (1), Weston (1), Westport (1), and Woodbridge (1).

For viral isolation from mosquitoes, frozen pools were thawed, triturated in tissue grinders or mortars with pestles in 1 to 1.5 ml of phosphate-buffered saline (“PBS”) containing 0.5% gelatin, 30% rabbit serum, antibiotic , and antimycotic. After centrifugation for 10 min at 520×g, 100 μl samples of each pool of mosquitoes were inoculated onto a monolayer of Vero cells grown in a 25-cm² flask at 37° C. in 5% CO₂. Cells were examined microscopically for cytopathologic effect for up to 7 days after inoculation.

For viral isolation from bird brain tissue samples, a 10% suspension of each sampled brain tissue was prepared in 1.5 ml of PBS by triturating with a mortar and pestle as described above for mosquito samples except that Alundum® was added to facilitate homogenization of tissue. Two to seven tissue samples from each brain were tested for virus as follows. Suspensions were centrifuged at 520×g for 10 min. The supernatant of each sample was then passed through a 0.22-μm filter before inoculation of a 100-μl sample onto a monolayer of Vero cells. Cells were grown in a 25-cm² flask at 37° C. in 5% CO₂ and examined for cytopathologic effect for up to 7 days after inoculation.

Viral isolates were tested in an ELISA against reference antibodies to six viruses, in three families, isolated from mosquitoes in North America. The antibodies were prepared in mice and provided by the World Health Organization Center for Arbovirus Research and Reference, Yale Arbovirus Research Unit, Department of Epidemiology and Public Health, Yale University School of Medicine. The antibodies were to Eastern Equine Encephalomyelitis and Highlands J, Cache Valley, LaCrosse, Jamestown Canyon, and St. Louis Encephalitis viruses.

### EXAMPLE II

### PCR Amplification of DNA Encoding the WN Virus Envelope Protein

We grew the Connecticut WN virus isolate 2741 (GenBank™ Accession No. AF206518) as described above in Vero cells which were subsequently scraped from the bottom of the flask and centrifuged at 4500×g for 10 min. We discarded the supernatant and extracted RNA from the pellet using the RNeasy® mini protocol (Qiagen), eluting the column twice with 40 μl of ribonuclease-free water. We used two microliters of each eluate in a 50-μl reverse transcription-polymerase chain reaction (RT-PCR) with the SuperScript® one step RT-PCR system (Life Technologies), following the manufacturer's protocol. We designed the PCR primers WN-233F (5′-GACTGAAGAGGGCAATGTTGAGC-3′; SEQ ID: 1) and WN-1189R (5′-GCAATAACTGCGGACYTCTGC-3′; SEQ ID: 2) specifically to amplify envelope protein sequences from WN viruses based on an alignment of six flavivirus isolates listed in GenBank™ [accession numbers: M16614; M73710; D00246; M12294; AF130362; AF130363]. We purified the PCR products with the QIAquick PCR Purification Kit® (Qiagen) following the manufacturer's protocol. The amplified DNA and sequenced by the Sanger method at the Keck Biotechnology Center at Yale University, New Haven, Conn. We confirmed that this sequence corresponded to the envelope protein encoding sequence by alignment with the envelope protein encoding sequence from other flavivirus isolates using the ClustalX 1.64B program [J. D. Thompson, et al., Nucleic Acids Res, 22, 4673 (1994)]. We further purified the resulting DNA fragments by electrophoresis on a 1% agarose gel, excised the DNA band, and isolated the DNA using the QIAquick Gel Extraction Kit® (Qiagen) following the manufacturer's protocol.

### EXAMPLE III

### Expression and Purification of Recombinant WN Virus Envelope Protein

We expressed the protein encoded by the DNA isolated in Example II in E. coli using the pBAD/TOPO™ ThioFusion Expression System® (Invitrogen). This system is designed for highly efficient, five minute, one step cloning of PCR amplified DNA into the pBAD/TOPO™ ThioFusion expression vector. Fusion protein expression is inducible with arabinose. Fusion proteins are expressed with thioredoxin (12 kDa) fused to the N-terminus, and a C-terminal polyhistidine tag. The polyhistidine tag allows fusion proteins to be rapidly purified by nickel affinity column chromatography. An enterokinase cleavage site in the fusion proteins can be used to remove the N-terminal thioredoxin leader.

We used the pBAD/TOPO ThioFusion Expression System® expression system to express and purify West Nile virus envelope protein following the manufacturer's protocol. Specifically, we added the PCR product obtained as described above to a reaction containing the pBAD/Thio-TOPO™ vector (1 μl) and sterile water added to a final volume of 5 μl. We incubated this reaction mix for five minutes at room temperature.

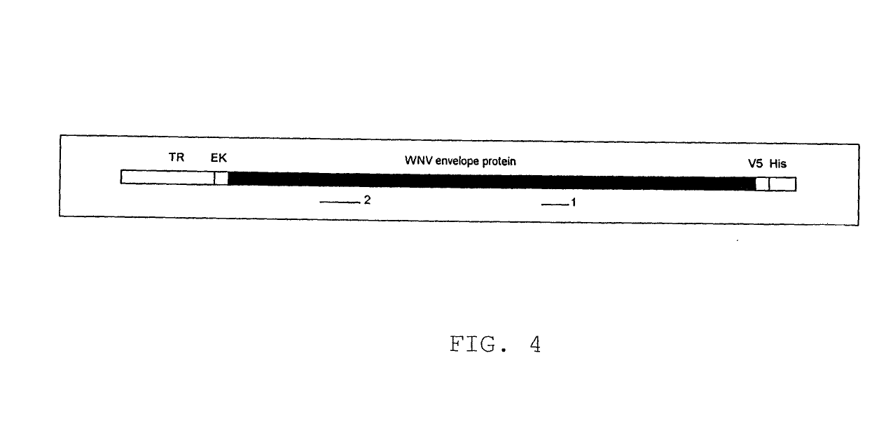

We transformed One Shot™ E. coli cells (Invitrogen) with the TOPO™ cloning reaction products by mixing the TOPO™ cloning reaction with competent cells, incubating the mixture on ice for 30 minutes and then heat shocking the cells for 30 seconds at 42° C. We added 250 μl of room temperature SOC medium to the cells and incubated at 37° C. for 30 minutes. We spread 50 μl of the transformation mixture on a prewarmed LB plate containing 50 μg/ml ampicillin and incubated overnight at 37° C. We then performed DNA sequence analysis to confirm that the thioredoxin-envelope protein fusion protein (TR-env; FIG. 4) coding sequences were correct.

To analyze expression of the recombinant TR-env protein, we grew E. coli containing the pBAD-TR-env expression vector in cultures at 37° C. with vigorous shaking to an OD₆₀₀=˜0.5. Prior to protein expression, i.e. at the zero point, we took an aliquot, centrifuged at maximum speed, removed the supernatant and stored the pellet on ice. We then induced protein expression with arabinose at a final concentration of 0.02% and grew the culture for an additional 4 hours. We centrifuged an aliquot of this arabinose-induced sample at maximum speed, removed the supernatant and placed on ice. We resuspended the uninduced and arabinose-induced pellets in sample buffer, boiled the sample for 5 minutes and analyzed by denaturing polyacrylamide (SDS-PAGE) gel and stained with Coomassie blue. We observed that the 71 kDa TR-env protein was the major protein found in the E. coli cells after arabinose induction.

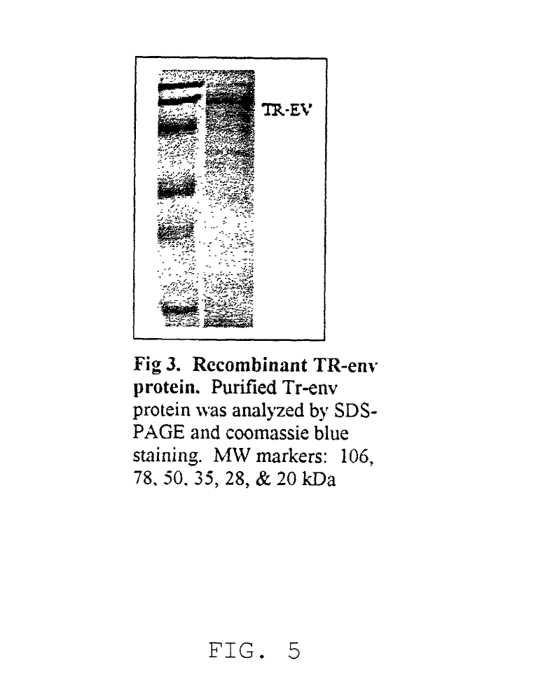

We lysed the induced E. coli cells by sonication, centrifuged, and purified the TR-env protein in the soluble supernatant with ThioBond™ phenylarsinine oxide resin (Invitrogen) following the manufacturer's protocol. The TR-env protein was bound to this affinity resin in a batch mode and then eluted with increasing concentrations of beta-mercaptoethanol. We ran the fractions on a denaturing polyacrylamide (SDS-PAGE) gel and stained with Coomassie blue. The procedure yielded highly purified recombinant TR-env fusion protein (FIG. 5).

In immunoblots, the TR-env protein was recognized by both anti-thioredoxin antibody (Invitrogen) and human sera from two individuals seropositive for antibodies to WN virus. The purified TR-env fusion protein, thus, contained an epitope recognized by antibodies induced by a natural WN virus infection.

Thioredoxin expressed from the pBAD/TOPO™ ThioFusion® expression vector was used as a negative control protein. The 16 kDa thioredoxin protein was expressed in E. coli and purified using ProBond™ metal-chelating affinity resin as described for the TR-env protein. Purified thioredoxin was recognized in immunoblots only by anti-thioredoxin antibody (Invitrogen) and not by human sera from two individuals seropositive for antibodies to WN virus.

Alternatively, we expressed and purified the WN virus envelope protein as a fusion protein with maltose binding protein (MBP). We amplified nucleotides 1-1218 of the WN virus E protein by PCR using the following primers which contain EcoRI and PstI restriction sites to facilitate subcloning: 5′-GAATTCTTCAACTGCCTTGGAATGAGC-3′ (SEQ ID NO: 6) and 5′-CTGCAGTTATTTGCCAATGCTGCTTCC-3′ (SEQ ID NO: 7). We digested the PCR product with EcoRI and PstI and cloned the resulting fragment into the pMAL™-c2X vector (New England Biolabs, Beverly, Mass.), creating a recombination fusion to the E. coli malE gene which encodes the maltose-binding protein (MBP). We grew DH5α containing the resulting plasmid to a concentration of 2×10⁸ cells/ml and added isopropyl-D-thiogalactopyranoside (IPTG) to a final concentration of 0.3 mM. We then incubated this culture for 2 hours at 37° C., harvested the cells by centrifugation at 4,000×g for 20 minutes and, lysed the cells by freezing overnight at −20° C. and then sonicating the cells for 10 minutes. We confirmed expression of a soluble 82 kDa MBP-env fusion protein in E. coli by SDS-PAGE analysis and Coomassie blue staining. We then purified the MBP-env fusion protein using a maltose-affinity column according to the manufacturer's instructions. We obtained 3 mg of protein from 250 ml of cell culture. We also purified MBP as a control following the same protocol.

We then used the MBP-env fusion protein to analyze sera for the presence of antibodies to the E protein. We boiled 2 μg of MBP-env fusion protein or MBP (control) protein in SDS-PAGE sample buffer (BioRad) containing 2% β-mercaptoethanol, ran the samples on a 10% SDS-PAGE gel and transferred the proteins to nitrocellulose membrane using a semi-dry electrotransfer apparatus (Fisher Scientific). We then probed the nitrocellulose membrane with sera from 5 patients with confirmed WN virus infection and sera from uninfected individuals. We incubated the membrane with the sera (1:100 dilution) for 1 hour, washed the membrane 3 times with Tris-buffered saline with Tween 20 (TBST) and added alkaline phosphatase-conjugated goat anti-human IgG (1:1,000 dilution; Sigma). We developed the blots with nitroblue tetrazolium and 5-bromo-4-chloro-3-indolyl phosphate (Kirkegaard & Perry Laboratories). The MBP-env fusion protein detected IgG antibodies to the E protein in western blots with sera from 5 humans with confirmed WN virus infection, but not in the control human sera. In essentially identical experiments, the MBP-env fusion protein also detects IgM antibodies to the E protein in western blots with sera from 5 humans with confirmed WN virus infection, and IgG and IgM antibodies with sera from 10 horses with confirmed WN virus infection, but not in control human or horse sera.

### EXAMPLE IV

### Validation of the Mouse Model of WN Virus Infection

Previous studies demonstrated that mice can be infected with the WN virus [A. H. Eldadah, et al., Am J Epidemiol, 86, pp. 765-75 (1967); S. Haahr, Acta Pathol Microbiol Scand, 74, pp. 445-47 (1968); L. P. Weiner, et al., J Hyg (Lond), 68, pp. 435-46 (1970); A. J. Johnson and J. T. Roehrig, J Virol, 73, pp. 783-6 (1999)]. All of these experiments used isolates of WN virus that have been recovered outside of the United States. We have extended these studies using WN virus isolates from Connecticut.

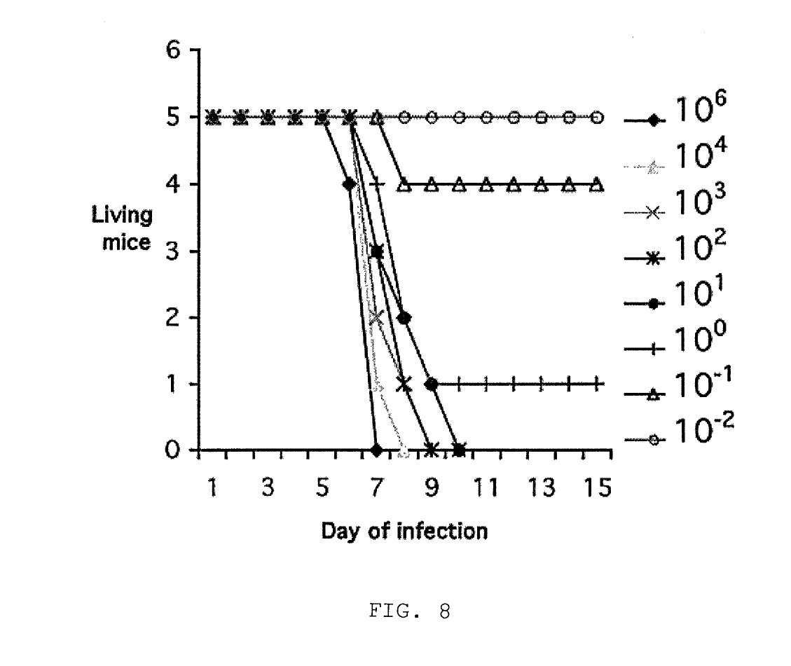

Following established protocols, we inoculated six-week-old C3H/HeN (C3H) mice intraperitoneally with a range of dilutions of the 2741 WN virus isolated as described in Example I. These dilutions corresponded to 10⁻²-10⁶ plaque-forming units of WN virus. We monitored the mice over a period of two weeks. The majority of the mice died after approximately 1-2 weeks. At 15 days surviving mice were sacrificed, and the blood, liver, spleen, and brain cultured for WN virus. Experiments used groups of five mice. We observed significant mortality at inoculation doses down to 1 plaque forming unit (10⁰) (FIG. 8). Our results showed that WN virus isolate 2741 from Connecticut is able to infect mice and that the course of infection is similar to murine disease that has been described for WN virus isolates from outside the U.S.

### EXAMPLE V

### Active Immunization with Purified TR-env and MBP-E Protein

We immunized two groups of three-week-old female C3H mice were immunized subcutaneously with 20 μg of purified TR-env or thioredoxin (TR) as a control antigen in Freund's adjuvant (complete for the first immunization on day 0; incomplete for booster immunizations on day 7 and day 14). We used an accelerated immunization schedule in this experiment because mice become less susceptible to West Nile virus encephalitis as they age. On day 21, we bled the mice, recovered the sera and conducted ELISA and immunofluorescence assays to determine the anti-envelope protein antibody titer. Immunized mice developed high titer anti-envelope protein antibodies.

We also actively immunized C3H mice with 20 μg MBP-env protein, or MBP protein as a negative control, as described above. Mice immunized with the MBP-env protein developed high titers of antibodies to the WN virus env protein. We then challenged mice with approximately 100 plaque-forming units of WN virus isolate 2741. We observed 100% survival of mice immunized with MBP-env protein compared to only 10% survival of mice immunized with MBP. [0174] We also actively immunize with the WNE 121-139 or the WNE 288-301 peptides and determine antibody titers as described above. We further challenge mice immunized with MBP-env, the WNE 121-139 peptide or the WNE 288-301 peptide with WN virus 2741 as described above and determine the degree of protection.

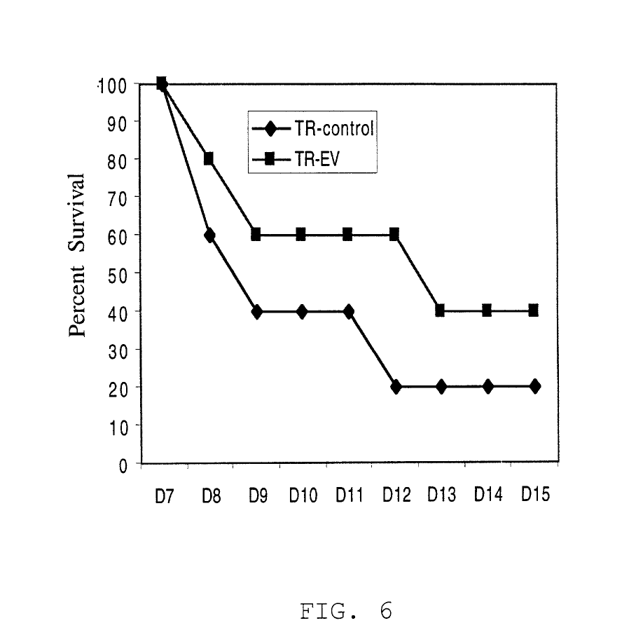

For an initial immunoprotection study, we challenged mice immunized with WN TR-env fusion protein or TR control protein as described above with 2.5×10⁶ plaque-forming units of WN virus isolate 2741 (intra-peritoneally). Mice were monitored daily for morbidity and mortality, and were euthanized if showing signs indicative of death (i.e. inappetence, coma, dehydration, or partial paralysis). The effects of WN virus infection became evident after seven days of infection. By day 15, two of five TR-env immunized mice survived compared to only one of the control mice. As indicated by the survival curves shown in FIG. 6, the control mice are more susceptible to the pathological effects of WN virus compared to mice immunized with WN TR-env fusion protein. This challenge experiment demonstrates that vaccination with envelope fusion protein can alter susceptibility to WN virus and is at least partially protective.

Having shown that TR-env elicits a partially protective immune response, we then optimize the protective response by varying the immunization conditions. For example, we alter the immunization schedule, adjuvants and dosing, or routes of administration and challenge dose. For example, as described in Example VII, challenge with 10 plaque-forming units in a passively immunized animal produced 80% protection. Those of skill in the art would recognize that envelope protein from other strains of WN virus can be used as the immunogen in the methods described above.

### Example VI

### Antibodies Generated to Purified TR-env Protein Recognize Synthetic Peptides

A. Selection of Peptides Representing Epitopes Recognized by Antibodies that Recognize the Envelope Protein

We prepared a structural model of the WN virus envelope protein using the 3D-PSSM Protein Fold Recognition (Threading) Web Server V 2.0 [L. A. Kelley et al., J Mol Biol, 299, pp. 499-520 (2000)]. We compared the WN virus structural model to a structural model of the tick-borne encephalitis (TBE) envelope glycoprotein protein soluble domain [F. A. Rey et al., Nature, 375, pp. 291-8 (1995)] using a three-dimensional position-specific scoring matrix (3D-PSSM). We used the model to design two synthetic peptides from WN virus envelope protein for testing as a vaccine. Peptide WNE 121-139 has sequence homology to a heparan sulfate binding domain found in the dengue virus envelope protein. Binding to target cells via heparan sulfate has been reported to play a role in flavivirus infectivity [Y. Chen et al., Nature Med, 3, pp. 866-871 (1997)]. An antibody that binds the WNE 121-139 peptide, thus, could alter WN virus binding to heparan sulfate and inhibit or prevent infection of target cells.

Based on the structural model, peptide WNE 288-301 appears to be a surface-exposed hinge region between domains I and II of the WN virus envelope protein. We also synthesized a negative control peptide, which we designated “random 288-301.” The control peptide has the same amino acid content as WNE 288-301, but in randomized sequence except for the N-terminal cysteine.

Peptides were synthesized on a Rainin Symphony™ instrument at a 50 μmol scale, purified by reverse phase HPLC and analyzed by MALDI mass spectroscopy. The peptide synthesis, purification and analysis were performed by the Keck Foundation Biotechnology Resource Laboratory at Yale University. The three peptides were conjugated to carrier proteins using Imject® maleimide activated ovalbumin and KLH (Pierce) following the manufacturer's instructions.

B. Synthetic Peptides are Recognized by Antibody Generated in Response to Immunization with Purified Tr-env

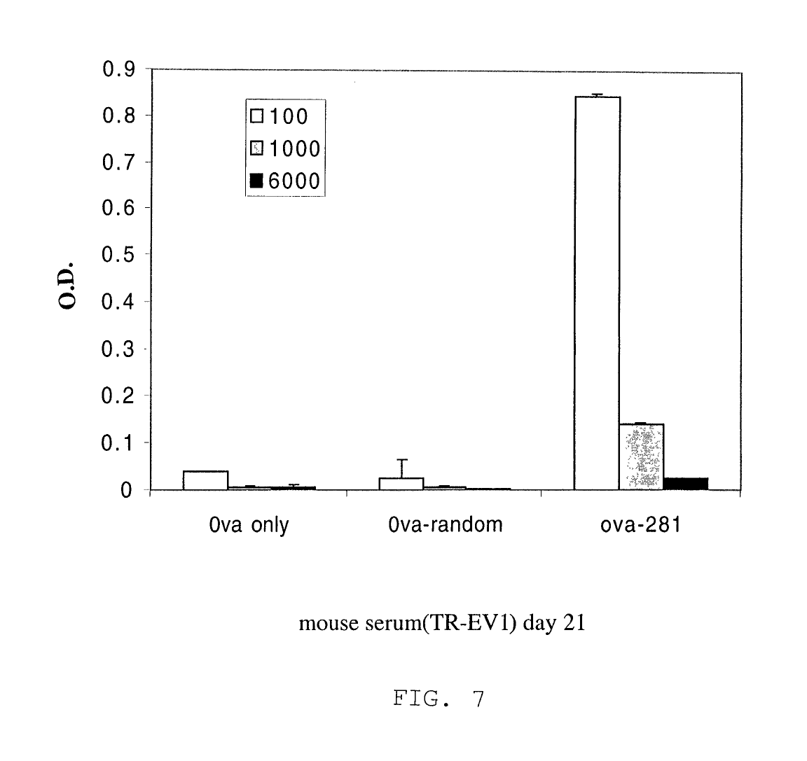

We tested sera from mice immunized with purified WN virus TR-env fusion protein as described in Example V for the presence of antibodies specific for the WNE 121-139 (SEQ ID: 3) peptide in an ELISA using the peptide as antigen (FIG. 7). Pooled antisera from TR-env immunized mice recognized peptide WNE-121-139 (SEQ ID: 3) conjugated to ovalbumin, but did not recognize the control peptide random-288-301 (SEQ ID: 5) or unconjugated ovalbumin. Pooled antisera from control thioredoxin immunized mice were negative in ELISAs with these antigens. Thus, immunization with the WN TR-env protein elicits antibodies that specifically recognize the peptide.

### EXAMPLE VII

### Passive Immunization

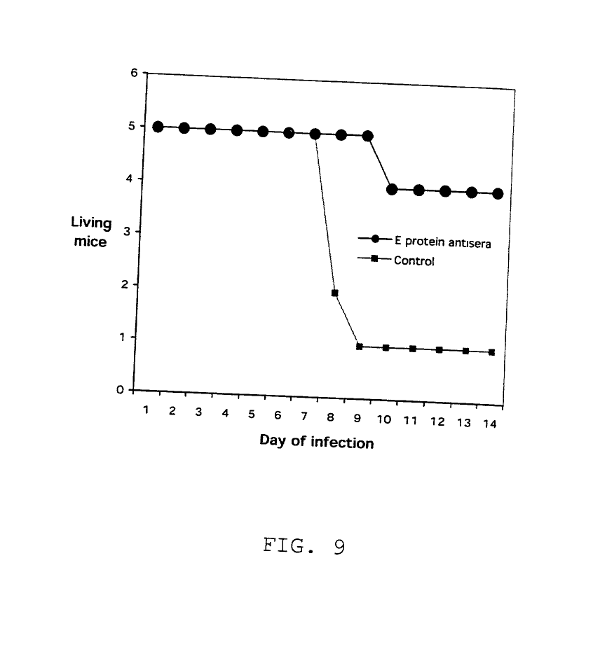

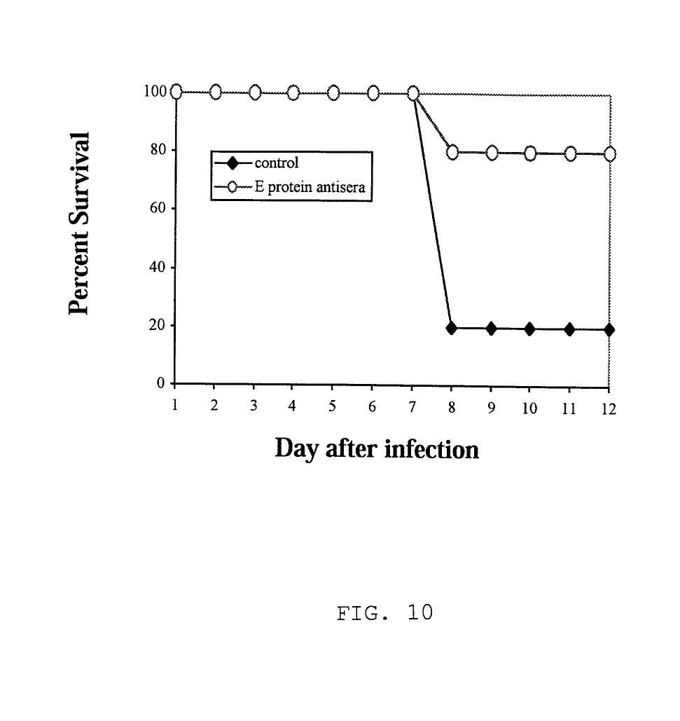

We prepared antiserum from C3H/HeN mice by immunizing in the back with 20 μg TR-env or TR (control) in 200 μl complete Freund's adjuvant. We then boosted the mice with 20 μg of antigen in 200 μl incomplete Freund's adjuvant at 2 and 4 weeks. We bled the mice ten days after the final immunization and stored the antisera at −20° C. We then passively immunized mice by intradermally injecting 100 μl of antisera (diluted 1:5 in PBS) that was pooled from 5 mice that had been actively immunized with the TR-env fusion protein. We also passively immunized a group of mice with TR antisera (control). We then challenged the passively immunized animals with approximately 10 plaque-forming units of the 2741 WN virus isolate 24 hours after immunization and evaluated immunity to WN virus as described in Example V. As shown in FIGS. 9 and 10, we observed protection of mice passively immunized with antisera directed to the E protein relative to control. This challenge experiment demonstrates that passive immunization with antisera to the envelope fusion protein can alter susceptibility to WN virus and is substantially protective.

### EXAMPLE VIII

### Analysis of Cross Protection

To determine the degree of protection against other WN virus isolates afforded by immunization with a polypeptide from one WN virus isolate (different WN virus isolates include, for example, WNV-NY1999, WNV-Cm-CT99, WNV-Crow-NJ99, WNV-Crow-NY99, WNV-C.pipiens-NY99, WNV-Eq.-NY99, WNV-HB709-NY99, WNV-HB743-NY99, WNV-USAMRIID99), we immunize mice with a polypeptide or fusion protein as described in Example V. We then challenge the immunized mice with a different WN virus isolate and monitor the mice as described in Example V. Alternatively, we perform a western blot using antibodies or antisera against one isolate of WN virus to detect polypeptides of another isolate.

### EXAMPLE IX

### Preparation of Protective Monoclonal Antibodies to WN Virus Polypeptides

To prepare antibodies to a WN virus polypeptide in one of the compositions of the invention, we immunize C3H mice subcutaneously with TR-env in complete Freund's adjuvant and boost with the same amount in incomplete Freund's adjuvant at 7 and 14 days. We immunize control animals in the same manner with either TR or bovine serum albumin (BSA).

Seven days after the last boost, we collect sera from the immunized animals and use it to hybridize to Western blots of SDS-PAGE gels of extract from WN virus-infected Vero cells or to the recombinant polypeptide. We detect binding with alkaline phosphatase goat-anti-mouse antibody developed with nitroblue tetrazolium and 5-bromo-4-chloroindoyl phosphate. Alternatively, we use the ECL™ kit (Amersham, Arlington Heights, Ill.) in which the secondary antibody, horseradish peroxidase-labeled goat anti-mouse antibody, can be detected.

To prepare a monoclonal antibody, we recover antibody producing cells from the spleens of the immunized animals, fuse the antibody producing cells with immortalized cells to produce hybridomas according to the methods of Kohler and Milstein. We screen the resulting hybridomas for specific binding to the Tr-env fusion protein described in this invention. Those of skill in the art will appreciate that polyclonal and monoclonal antibodies specific of the other WN virus polypeptides in the compositions of the invention may be prepared using the methods described herein.

A protective antibody, including a monoclonal antibody, may be identified, for example, by passively immunizing mice with the antibody, challenging the mice with WN virus and monitoring infection in the mice.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A composition for treating, inhibiting or preventing West Nile (WN) virus infection or disease, comprising one or more isolated and substantially purified WN virus polypeptides or an immunogenic fragment thereof and a pharmaceutically acceptable carrier.

2. The composition according to claim 1, wherein the WN virus polypeptide is a WN virus envelope (E) protein or an immunogenic fragment thereof.

3. The composition according to claim 2, wherein the WN virus E protein or fragment is from isolate WN 2741.

4. The composition according to claim 3, wherein the amino acid sequence of the WN virus E protein or fragment is the amino acid sequence encoded by the E protein encoding DNA sequence of Genbank accession No. AF 206518, or a fragment thereof.

5. The composition according to claim 1, wherein said WN virus polypeptide or fragment comprises a sequence with homology to a heparan sulfate binding domain.

6. The composition according to claim 4, wherein said fragment is the WNE 121-139 peptide (SEQ ID NO: 3).

7. The composition according to claim 4, wherein said fragment is the WNE 288-301 peptide (SEQ ID NO: 4).

8. The composition according to claims 6 or 7, wherein said WN virus peptide is joined to keyhole limpet hemocyanin (KLH).

9. The composition according to claim 1, further comprising an adjuvant.

10. The composition according to any one of claims 1-7 and 9, wherein said WN virus polypeptide or fragment is part of a fusion protein.

11. The composition according to claim 10, wherein said fusion protein comprises thioredoxin or maltose binding protein.

12. The composition according to claim 2 comprising at least one additional WN virus polypeptide or an immunogenic fragment thereof.

13. The composition according to claim 12, wherein said additional WN virus polypeptide or fragment is from the same isolate of WN virus as the E protein.

14. The composition according to claim 12, where said additional WN virus polypeptide or fragment is from a different isolate of WN virus than the E protein.

15. The composition according to claim 12, wherein said additional WN virus polypeptide is selected from the group consisting of: a capsid (C) protein, a membrane (M) protein and a non-structural (NS) protein, or an immunogenic fragment thereof.

16. The composition according to claim 15 wherein said additional WN virus polypeptide is an NS protein or an immunogenic fragment thereof.

17. The composition according to claim 2 comprising at least one additional WN virus E polypeptide, or an immunogenic fragment thereof, wherein said WN virus E polypeptides are from different isolates of WN virus.

18. The composition according to any one of claims 1-17, further comprising an immunogenic component from another arthropod-borne pathogen.

19. The composition according to claim 18, wherein said arthropod-borne pathogen is a flavivirus.

20. A composition comprising an antibody that specifically binds a WN virus polypeptide or an immunogenic fragment thereof, wherein said antibody inhibits or lessens the severity of WN virus infection or disease.

21. The composition according to claim 20, wherein said antibody specifically binds a WN virus E protein or a fragment thereof.

22. An antibody or an antigen-binding portion thereof that specifically binds a WN virus E protein or an immunogenic fragment thereof.

23. The antibody or an antigen-binding portion thereof according to claim 22, wherein the E protein or fragment is from WN virus isolate 2741.

24. The antibody or an antigen-binding portion thereof according to claim 23, wherein said fragment is WNE 121-139 peptide (SEQ ID NO: 3).

25. The antibody or an antigen-binding portion thereof according to claim 23, wherein said fragment is WNE 288-301 peptide (SEQ ID NO: 4).

26. The antibody or an antigen-binding portion thereof according to claim 23, which specifically binds a heparan sulfate binding domain of a WN virus E protein.

27. The antibody or an antigen-binding portion thereof according to any one of claims 22-26, which is a monoclonal antibody.

28. The antibody or an antigen-binding portion thereof according to any one of claims 22-27, wherein said antibody or antigen-binding portion thereof inhibits or lessens the severity of WN virus infection or disease.

29. An antibody or an antigen-binding portion thereof produced by immunizing a non-human mammal with a polypeptide according to any one of claims 43-49.

30. A diagnostic kit comprising at least one polypeptide according to any one of claims 43-49.

31. The kit according to claim 30, wherein said E protein or fragment is from WN virus isolate 2741.

32. The kit according to claim 31, wherein the amino acid sequence of the WN virus E protein or fragment is the amino acid sequence encoded by the E protein encoding DNA sequence of Genbank accession No. AF 206518, or a fragment thereof.

33. The kit according to any one of claims 30-32, wherein said E protein or fragment is part of a fusion protein.

34. A nucleic acid molecule comprising a nucleotide sequence encoding a fusion protein, said fusion protein comprising a WN virus E protein or an immunogenic fragment thereof.

35. The nucleic acid molecule according to claim 34, wherein said E protein fragment is the WNE 121-139 peptide (SEQ ID NO: 3).

36. The nucleic acid molecule according to claim 34, wherein said E protein fragment is the WNE 288-301 peptide (SEQ ID NO: 4).

37. The nucleic acid molecule according to any one of claims 34-36, wherein the nucleic acid sequence is operably linked to an expression control sequence.

38. A host cell comprising the nucleic acid molecule according to any one of claims 33-37.

39. A method for producing a polypeptide encoded by a nucleic acid molecule according to any one of claims 33-37, comprising the step of culturing the host cell according to claim 38.

40. A method for treating, inhibiting or preventing WN virus infection or disease comprising the step of administering a compositions according to any one of claims 1-19.

41. A method for treating, inhibiting or preventing WN virus infection or disease comprising the step of administering a composition according to claim 20 or 21.

42. A method for treating, inhibiting or preventing WN virus infection or disease comprising the step of administering an antibody or an antigen-binding portion thereof according to any one of claims 22-29.

43. An isolated and purified polypeptide comprising a WN virus envelope (E) protein or an immunogenic fragment thereof.

44. The polypeptide according to claim 43, which is a fusion protein.

45. An isolated polypeptide, consisting essentially of a WN virus envelope (E) protein or an immunogenic fragment thereof.

46. An isolated polypeptide, consisting of a WN virus envelope (E) protein or an immunogenic fragment thereof.

47. The polypeptide according to any one of claims 43-46, wherein said WN virus E protein is from WN virus isolate 2741.

48. The polypeptide according to any one of claims 43-47, wherein said polypeptide is the WNE 121-139 peptide (SEQ ID NO: 3).

49. The polypeptide according to any one of claims 43-47, wherein said polypeptide is the WNE 288-301 peptide (SEQ ID NO: 4).

50. A method for detecting WN virus infection comprising the step of contacting a sample from a subject suspected of having said infection with an isolated and substantially purified polypeptide according to any one of claims 43-49.

51. A method for detecting a protective immune response in a subject comprising the step of contacting a sample from said subject with a polypeptide according to any one of claims 43-49.

52. A method for detecting WN virus infection comprising the step of contacting a sample from a subject suspected of having said infection with the antibody or an antigen-binding portion thereof according to any one of claims 22-29.

53. A method for treating, inhibiting or preventing WN virus infection or disease comprising the step of administering a polypeptide according to any one of claims 43-49.

54. A method for identifying a protective WN virus polypeptide comprising the steps of:
a) immunizing a C3H mouse with an WN virus polypeptide or fragment thereof;
b) challenging the immunized mouse produced in step a) with WN virus; and
c) identifying a WN virus polypeptide that confers protection against WN virus infection or disease.

55. A method for identifying an protective antibody that specifically binds a WN virus polypeptide, comprising the steps of:
a) passively immunizing a C3H mouse with an antibody that specifically binds a WN virus polypeptide;
b) challenging the immunized mouse produced in step a) with WN virus; and
c) identifying an antibody that confers protection against WN virus infection or disease.
